(19) United States
(12) Patent Application Publication
(10) Pub. No.: US 2010/0279393 A1
(43) Pub. Date: Nov. 4, 2010

(54) MICRO CHIP DEVICE

(76) Inventor: Taisuke Hirono, Chofu-shi (JP)

Correspondence Address: Law Offices of Robert F. Zielinski, LLC, 1518 Walnut Street, Suite 1706, Philadelphia, PA, 19102, US

(21) Appl. No.: 12/698,425

(22) Filed: Feb. 2, 2010

(30) Foreign Application Priority Data
May 2, 2009 (JP) 2009-024522

(51) Int. Cl. C12M 1/34

(52) U.S. Cl. 435/287.1

(57) ABSTRACT

In FIG. 2(a), both laminar flows, solution including fine grains and buffer solution, are supplied in a microchannel in a micro chip device, and gel including drug is supplied at a position contacting with the buffer solution. The fine grains in the solution including fine grains starts to react after contacting with the gel including drug, but the reaction does not start since the drug is separated from the buffer solution in FIG. 2 (c). If supply of the buffer solution is stopped in the above-mentioned state, the solution including fine grains and the gel including drug are contacted and the reaction between the fine grains and the drug can be observed at a fixed point. In such a device, it is sufficient to supply the gel including drug from the supply passage without coating the drug on a wall face of the microchannel and the drug coating operation can be omitted thereby.

### CROSS REFERENCE TO RELATED APPLICATION

The present disclosure relates to subject matter contained in Japanese patent application No.2009-024522 filed on Feb. 5, 2009, the disclosure of which is expressly incorporated herein by reference in its entirety.

### BACKGROUND OF THE INVENTION

This invention relates to a micro chip device for observing a process wherein specific fine grains react with specific drug.

In the past, processes of a reaction between some specific fine grains and specific drug have been observed, and a micro chip device is used as a device for inducing the reaction (see a Japanese patent application publication 2007-315753).

FIG. 3 is a typical view showing an example of a structure of a conventional micro chip device for a reaction of fine grains, and FIG. 4 is a typical view for explaining the operations. A reference mark B₁ in the figure denotes a channel of solution D₁ having no fine grain which does not react with the above-mentioned drug (“the buffer solution” hereinafter), and a reference mark P₁ denotes a pump for supplying the buffer solution D₁. A reference mark B₂ denotes a channel of solution including fine grains D₂, and a reference mark P₂ denotes a pump for supplying the solution D₂. Both channels B₁ and B₂ meet on a downstream side, and a microchannel B₃ communicates with such a meeting portion C so that solutions from both channels B₁ and B₂ (that is, the buffer solution D₁ and the solution including fine grains D₂) flow down forming two layers without mixing with each other. Besides, a reaction portion (a portion on which drug is coated, see a reference mark G of FIG. 4 (a), (b)) which reacts with only the solution including fine grains D₂ and does not react with the buffer solution D₁ is located at a wall face of the microchannel B₃. Then, the reaction portion G is contacted with the solution including fine grains D₂ by moving an interface E from a state of FIG. 4 (a) to a state of FIG. 4 (b) through adjustment of layer widths W₁, W₂ of both solutions D₁ and D₂ in the microchannel B₃ with the pumps P₁ and P₂ in order to induce the reaction of the fine grains.

However, the above-mentioned prior art has such a problem that it is difficult to form the reaction portion by coating drug on the wall face of the microchannel.

The object of the invention is to provide a micro chip device for solving the above-mentioned problem.

### SUMMARY OF THE INVENTION

One aspect of the invention is a micro chip device, comprising:

a microchannel wherein supplied solution flows down in a state of a laminar flow;

a first supply portion for supplying solution including fine grains in said microchannel in a state of a laminar flow, said first supply portion connected with said microchannel;

a second supply portion for supplying gel including drug for reaction of said fine grains so as to face said microchannel, said second supply portion connected with said microchannel;

a third supply portion for supplying buffer solution in a state of a laminar flow between said solution including fine grains and said gel including drug, said third supply portion connected with said microchannel; and

means for adjusting buffer solution for adjusting amount of supply of said buffer solution from said third supply portion to said microchannel.

According to this aspect of the invention, it is not necessary to coat drug on a wall face of the microchannel, and the coating operation can be avoided thereby.

Another aspect of the invention is the micro chip device, wherein said solution including fine grains is solution including blood platelets, and said gel including drug is gel mixing collagen therein.

Another aspect of the invention is the micro chip device, wherein a concentration of collagen in said gel including drug is 1 μg/ml through 20 μg/ml.

According to both aspects of the invention, the coagulated mass generated by contacting blood platelets and collagen with each other is not flowed downstream due to an operation of holding the gel including collagen and in such a state the coagulation reaction proceeds, so that the coagulation reaction of blood platelets can observed at a fixed position.

### BRIEF DESCRIPTION OF THE DRAWINGS

FIG. 1 is a typical view for explaining one instance of the whole structure of a micro chip device according to the invention;

FIG. 2 is a typical view for explaining operations of the micro chip device according to the invention;

FIG. 3 is a typical view of a conventional micro chip device for reaction of fine grains; and

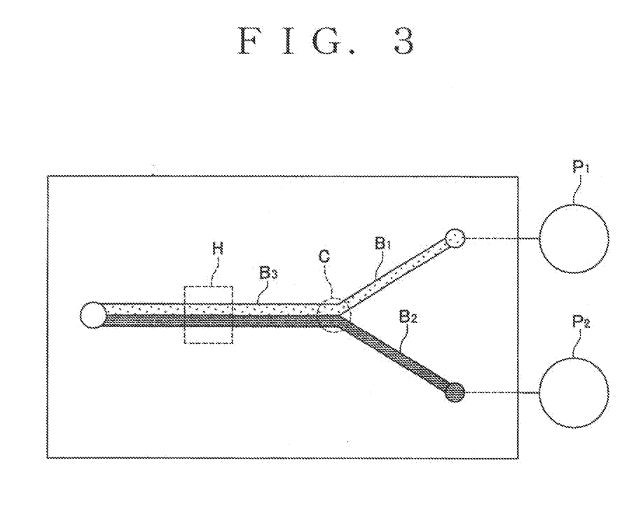

FIG. 4 is a typical view for explaining operations of the device as shown in FIG. 3.

### DESCRIPTION OF THE PREFERRED EMBODIMENTS

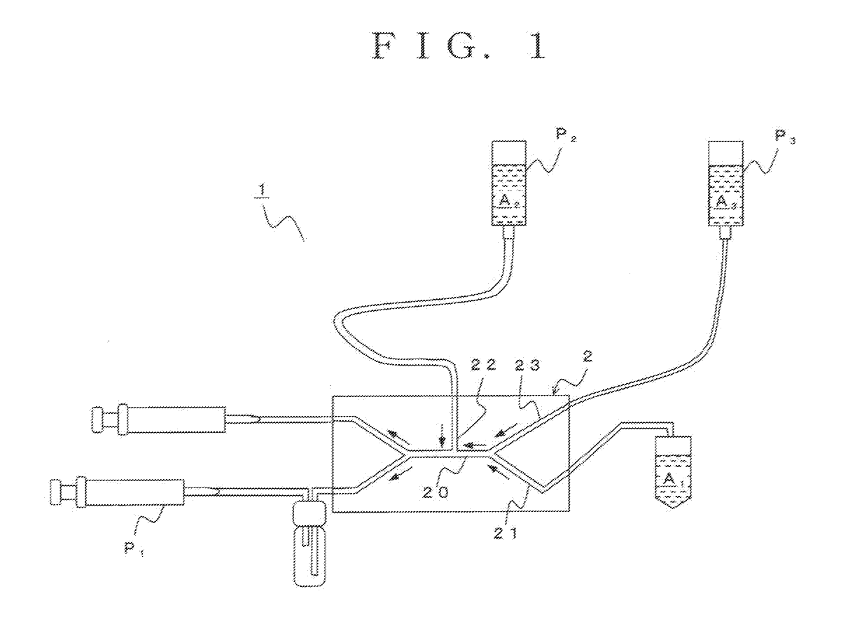

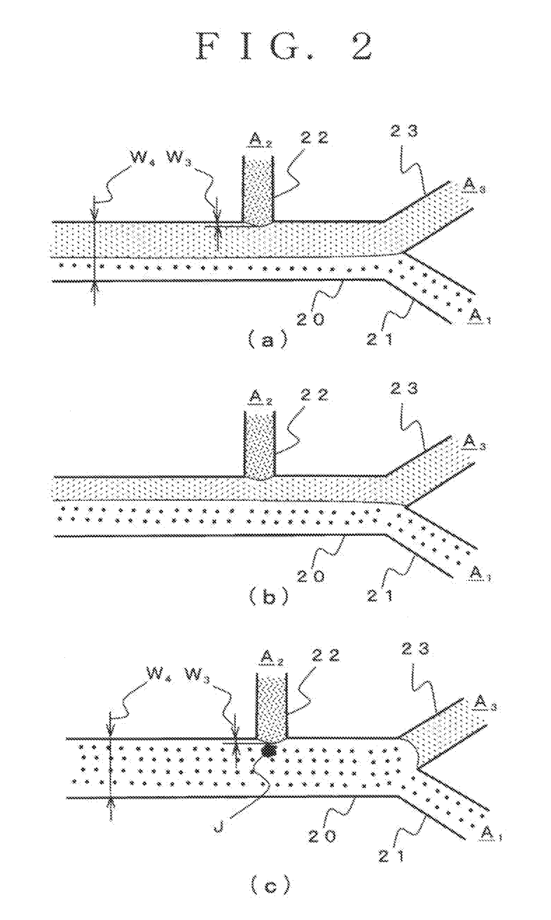

An embodiment of the invention is now mentioned, referring to FIGS. 1 and 2.

A micro chip device according to the invention is denoted with a reference numeral 1 in FIG. 1, and has a channel 20 with a micro section (“the micro channel” hereinafter) , and the microchannel 20, which Reynolds number is extremely low, is formed such that supplied solutions flow down in a state of a laminar flow.

A first supply portion 21 for supplying solution including fine grains A₁ (solution including some specific fine grains) is connected with an upper stream side of the microchannel 20 (right side in FIG. 1), and the solution including fine grains A₁ supplied from the first supply portion 21 flows down in the microchannel 20 in a state of a laminar flow. And, a second supply portion 22 for supplying gel including drug for reaction of the fine grains (“the gel including drug” hereinafter) A₂ so as to face the microchannel 20 is connected with the microchannel 20 (more detailedly speaking, the lower stream side rather than a portion connecting with the first supply portion 21). Besides, a third supply portion 23 for supplying buffer solution A₃ (solution which does not react with the solution including fine grains A₁ or the gel including drug A₂) is connected with the microchannel 20 (more detailedly speaking, the upper stream side rather than a portion connecting with the second supply portion 22) so that the buffer solution A₃ in a state of a laminar flow flows between the solution including fine grains A₁ and gel including drug A₂ so as not to obstruct both solutions (see FIG. 2 (a)).

The above-mentioned first supply portion 21 is a supply passage (channel) having a micro section where the solution including fine grains A₁ flows in a state of a laminar flow, for example, and the third supply portion 23 is a supply passage (channel) having a micro section where the buffer solution A₃ flows in a state of a laminar flow, for example. Preferably, the first supply portion 21, the third supply portion 23 and the microchannel 20 are located so as to form an almost Y character as shown in the figure if both first supply portion 21 and the third supply portion 23 are the supply passages. Preferably, the first through third supply portions 21, 22 and 23 and the microchannel 20 are formed inside the micro chip device 2. In FIGS. 1 and 2, the supply passage of gel including drug (the second supply portion) 22 is provided almost perpendicular to the microchannel 20, but may have the other structure. That is, the second supply portion 22 may be inclined.

Means for adjusting buffer solution P₃ that adjusts supply amount of the buffer solution A₃ from the third supply portion 23 to the microchannel 20 may be provided. Means for adjusting buffer solution P₃ is not a pressure-control pump, but may be a voltage-control pump, such as an electro-osmotic pump, and such a pump may be connected with the third supply portion 23. The latter, the voltage control pump is suitable for adjusting supply amount of buffer solution since a fine flow control is possible although in the former, the pressure-control pump, a pump actuator is widely displaced by a slight change of voltage and it is difficult to finely control the flow.

Besides, means for adjusting solution including fine grains P₁ that adjusts supply amount of the solution including fine grains A₁ from the first supply portion 21 to the microchannel 20 may be provided. Means for adjusting solution including fine grains P₁ is a pressure-control pump, such as a syringe pump, for instance. In case of such a pressure-control pump, distribution of flow velocity is a Poisson distribution, and an accurate analysis is possible since movement positions of fine grains can be computed by the Navier-Stokes equations.

The second supply portion 22 may be a supply passage having a micro section (channel). And, means for adjusting drug P₂ may be provided so as to adjust amount of projection W₃ (will be mentioned hereinafter) of the gel including drug A₂ from the second supply portion 22 to the microchannel 20. Means for adjusting drug P₂ may be a voltage-control pump, such as an electro-osmotic pump, for instance, similar to means for adjusting buffer solution P₃.

Solution including blood platelets (platelet-rich plasma) may be used as the solution including fine grains, and gel including a coagulant, such as collagen, may be used as the gel including drug A₂. In such a case, coagulation reaction of blood platelets can be observed at a fixed point. Preferably, a concentration of collagen in the gel is 1 μg/ml through 20 μg/ml.

Subsequently, operations of the invention are now mentioned.

In order to induce a reaction of fine grains, means for adjusting solution including fine grains P₁, means for adjusting drug P₂ and means for adjusting buffer solution P₃ are properly adjusted so that the solution including fine grains A₁ and the buffer solution A₃ flow down in the microchannel 20 in a state of a laminar flow and the gel including drug A₂ faces the microchannel 20 (see FIG. 2 (a)). “Facing” means such a state that the gel including drug A₂ faces an inside of the microchannel 20 (that is, the inside of the channel filled with the solution including fine grains A₁ or the buffer solution A₃), and concretely speaking, “facing” means both states, such a state that the gel including drug A₂ projects inside the microchannel 20 predetermined amount as shown by a reference mark W₃ of FIG. 2 (a), and a such a state that the gel including drug A₂ does not project inside the microchannel 20 (that is, the state that amount of projection W≈3). In the above-mentioned state, the supply of the buffer solution A₃ is stopped and a width of the layer of the solution including fine grains A₁ is increased by adjusting means for adjusting buffer solution P₃ and the like (see FIG. 2(b), (c)). At the result, the solution including fine grains A₁ contacts with the gel including drug A₂, and the fine grains react with the drug. In a case where the solution including blood platelets is used as the solution including fine grains and the gel including collagen is used as the gel including drug A₂, the blood platelets react with the collagen in the gel and a coagulated mass generates. The thus generated coagulated mass remains there, and the coagulation reaction proceeds without downstream flowing the coagulated mass from the location (see a reference mark J of FIG. 2 (c)). In such a case, the supply of the buffer solution A₃ by only means for adjusting buffer solution P₃ may be stopped without operating means for adjusting solution including fine grains P₁ and means for adjusting drug P₂. The inventor of the invention has confirmed that the width of the layer of the solution including fine grains A₁ increased as shown in FIG. 2(c) even in such a case.

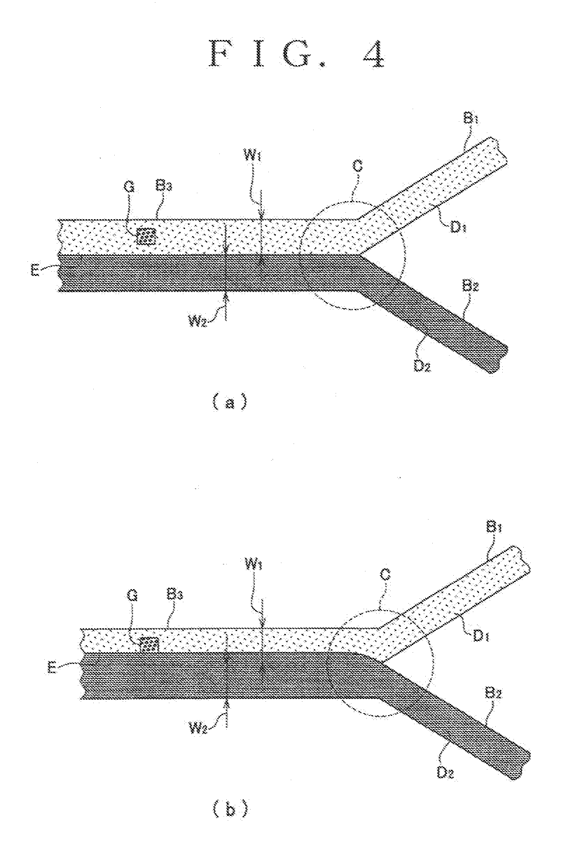

According to the invention, drug is mixed in the gel A₂ and the form of the drug can be held even if the buffer solution A₃ or the solution including fine grains A₁ flows in the microchannel 20, and the observation of the drug at a predetermined point is possible thereby. Besides, it is not necessary to coat drug (reaction portion G) on a wall face of the microchannel as shown in FIG. 4, and a coating operation can be avoided. Furthermore, in case where the solution including blood platelets is used as the above-mentioned solution including fine grains and the gel including collagen is used as the gel including drug A₂, a reaction of a coagulated mass generated by contacting blood platelets and collagen proceeds due to an operation of holding the gel including collagen without downstream flowing such a mass, and a coagulation reaction of blood platelets can be observed at a fixed point.

The present invention has been explained on the basis of the example embodiments discussed. Although some variations have been mentioned, the embodiments which are described in the specification are illustrative and not limiting. The scope of the invention is designated by the accompanying claims and is not restricted by the descriptions of the specific embodiments. Accordingly, all the transformations and changes within the scope of the claims are to be construed as included in the scope of the present invention.

1. A micro chip device, comprising:
a microchannel wherein supplied solution flows down in a state of a laminar flow;
a first supply portion for supplying solution including fine grains in said microchannel in a state of a laminar flow, said first supply portion connected with said microchannel;
a second supply portion for supplying gel including drug for reaction of said fine grains so as to face said microchannel, said second supply portion connected with said microchannel;
a third supply portion for supplying buffer solution in a state of a laminar flow between said solution including fine grains and said gel including drug, said third supply portion connected with said microchannel; and
means for adjusting buffer solution for adjusting amount of supply of said buffer solution from said third supply portion to said microchannel.

2. The micro chip device according to claim 1, wherein said solution including fine grains is solution including blood platelets, and said gel including drug is gel mixing collagen therein.

3. The micro chip device according to claim 2, wherein a concentration of collagen in said gel including drug is 1 μg/ml through 20 μg/ml.
